(19) United States
(12) Patent Application Publication
(10) Pub. No.: US 2011/0243857 A1
(43) Pub. Date: Oct. 6, 2011

(54) SMART CONTRAST AGENTS FOR MRI IMAGING

(75) Inventors: Geraldine Gouhier, Criquetot Sur Ouville (FR); Francois Estour, Rouen (FR)

(73) Assignee: UNIVERSITE DE ROUEN, Mont-saint-Aignan (FR)

(21) Appl. No.: 13/140,360

(22) PCT Filed: Dec. 18, 2009
(86) PCT No.: PCT/EP2009/067587

§ 371 Date: Jun. 16, 2011

Related U.S. Application Data
(60) Provisional application No. 61/139,255, filed on Dec. 19, 2008.

(51) Int. Cl. A61K 49/10; C07F 5/00; C12Q 1/02; C07K 2/00

(52) U.S. Cl. 424/9.35; 534/16; 435/29; 530/322

(57) ABSTRACT

A contrast agent for MRI imaging includes: (a) one cyclodextrin, whose truncated-cone-shaped structure defines a central axis, a first and a second openings along the axis, (b) one paramagnetic element, located on the cyclodextrin axis, outside the structure and proximate to the first opening, (c) one or several coordination ligand(s) of the paramagnetic element which coordinate(s) the paramagnetic element, (d) one arm covalently bound to the cyclodextrin, proximate to the second opening, which is able to form an inclusion complex with the cyclodextrin.

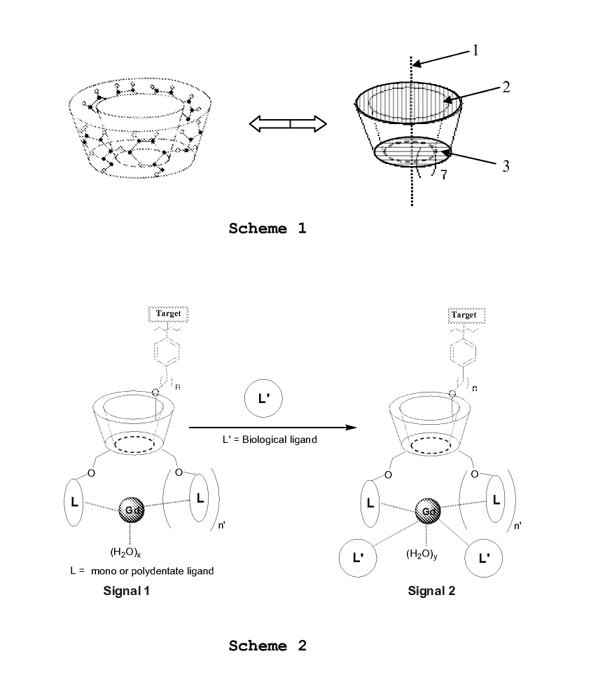

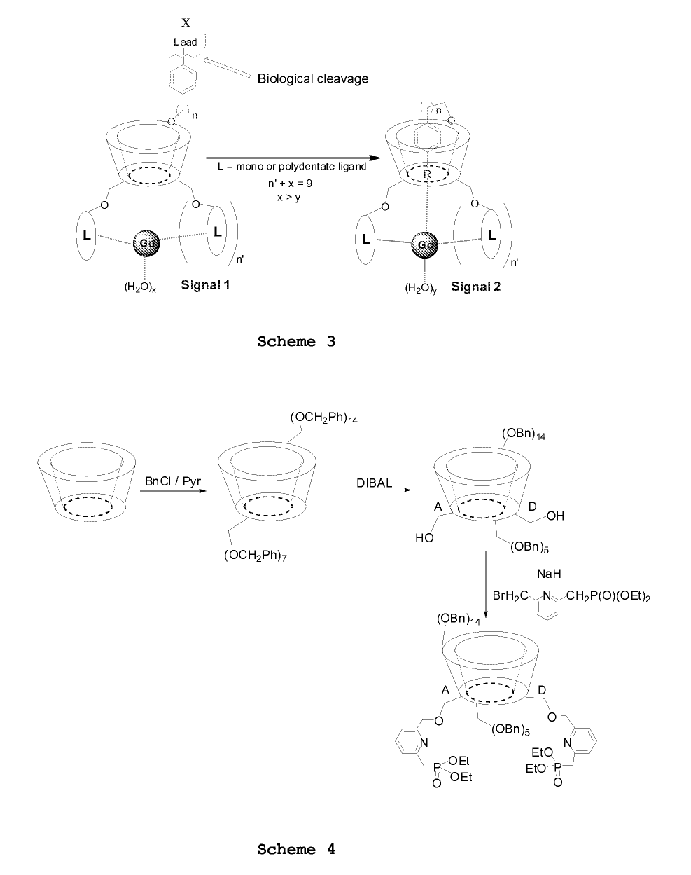

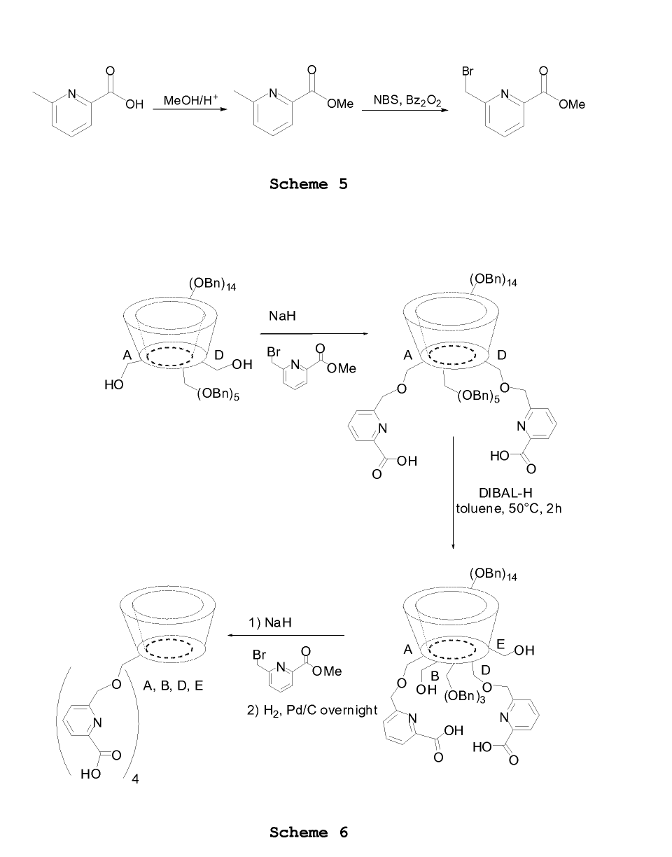

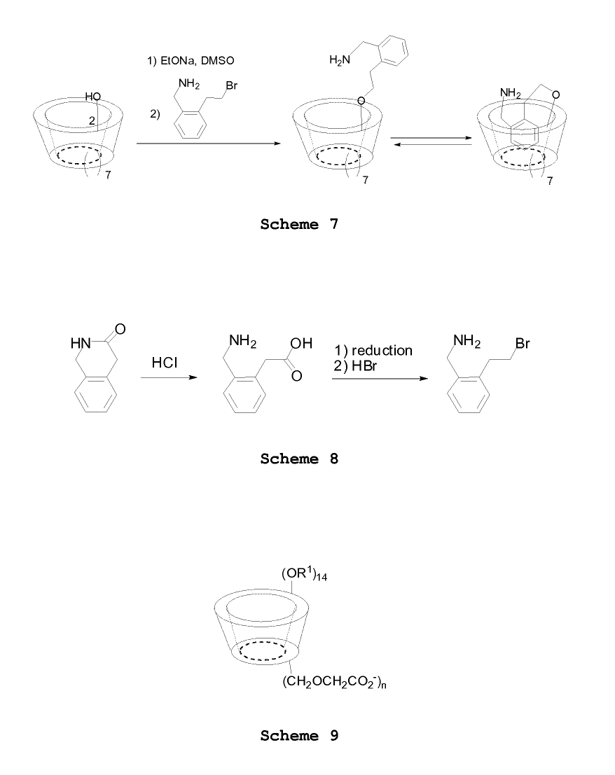

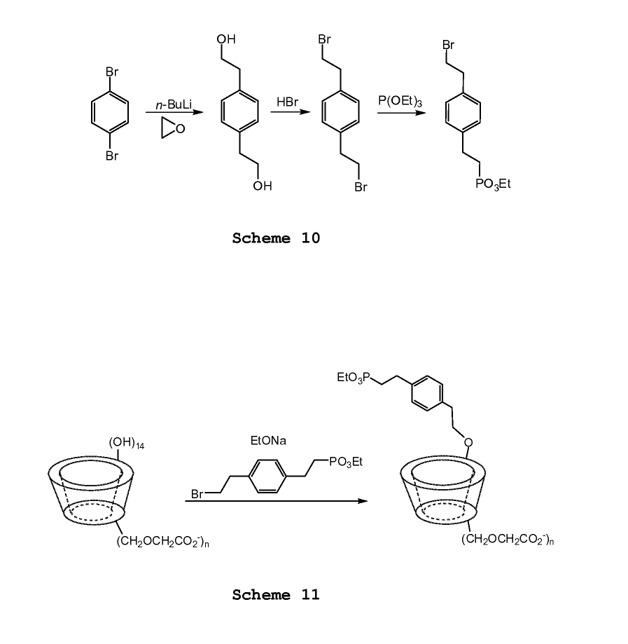

The invention relates to contrast agents for MRI imaging. Further, the invention relates to bioactivable contrast agents for MRI imaging. These contrast agents can be activated, i.e. they can produce the expected MRI signals once the phenomenon of interest has occurred and they can display enhanced specificity. They allow the in vivo detection of biological phenomena.

### BACKGROUND

### About MRI and CAs

Owing to its excellent resolution, magnetic resonance imaging (MRI) is currently considered as the technique of choice in modern diagnostic investigations. More than 35% of clinical scans are presently performed with the administration of contrast agents (CAs), affording a better discrimination between pathological and normal tissues. The most frequently used CAs for MRI are gadolinium(III) chelates, whose high paramagnetism (seven unpaired electrons) can yield to a strong enhancement of the water proton relaxation rates in the tissues in which they distribute. The examination time is reduced, the intensity of the signal is increased, and therefore the quality of imaging of the partition of water of the patient is improved.

In comparison with iodine CAs, the allergenic reactions with gadolinium are extremely rare. Since the free lanthanide is poorly tolerated, it must be coordinated by a strongly binding ligand that occupies most of the available coordination sites, leaving one or two sites free for water molecules, which relaxation rate is detectable by MRI. This kind of chelate stabilizes the complex that becomes non toxic, chemically inert and stable in a living organism. Thus, the exchanges with endogenous metal ions (Zn²⁺, Cu²⁺, Fe²⁺, Ca²) is avoided. This tracer is excreted through urine under its complex form (half-life 80-100 min.).

The first generation of CAs, introduced in 1988, and currently used in clinical diagnoses are highly hydrophilic agents. The typical example of this class is Gd-DPTA (DPTA: diethylene-triamine-pentaacetic acid; r₁=3.8 mM¹s⁻¹). It has been used in hundreds of millions of MRI scans up to now. Gd-DTPA that has a non specific extracellular distribution, has particularly demonstrated its utility in detecting abnormalities in the blood-brain barrier and in renal clearance. It is commonly administered at doses of ca. 0.1 mmol/kg. This means that in order to give enough contrast, Gd-DTPA has to reach concentrations of the order of 50-100 microM. In the case of MRI, has a relatively low sensibility in comparison with tracers used in others diagnostic techniques (SPECT, PET, Optical imaging). However, in spite of their high sensitivities, these approaches suffer from poor image resolution.

To compensate for this drawback, it is necessary:

(i) to develop systems endowed with high relaxivities;
(ii) to accumulate at the target site a large number of CA units that can recognize specific cell-membrane markers of a given disease.

The measurement of the relaxivity of a gadolinium complex as a function of magnetic field strength generates a nuclear magnetic resonance dispersion profile (NMRD) that has been modelled to provide reliable estimates of τₘ (the water exchange rate), τᵣ (the reorientational correlation time) and τₛ (the electronic spin relaxation time). The main determinant of the relaxation enhancement is represented by the overall time needed for molecular reorientation.

Efficiency of the contrast agent can be improved by acting upon the three parameters of relaxivity:

(i) increase of τₘ, the water exchange rate, thanks to the presence of more water molecules. The hydration number plays an important role in determining relaxivity;
(ii) increase of τᵣ, the reorientational correlation time;
(iii) modifying of τₛ/the electronic spin relaxation time, by introducing suitable electron donor substituents in order to reduce the positive charge of the metal centre and modify the water exchange dynamics from exchangeable protons hydrogen-bonded to polar groups present on the surface of the Gd(III) complex. Furthermore, the presence of negative complex reduces the affinity towards anionic in vivo substrates.

About the Use of CDs:

It has been noticed that, with the magnetic fields commonly used in MRI (0.2-17.6T), an enhancement of the relaxation rate of water protons is observed if the paramagnetic complex is part of a macromolecular system, due to the lengthening of τᵣ of the supramolecular adduct. This approach has been exploited either through the synthesis of covalent conjugates with macromolecular system like proteins, dendrimers, and polypeptides or through non-covalent interactions between a suitable functionalized chelate and proteins (essentially Human Serum Albumin) or micelles.

In particular when cyclodextrins (CDs) are used in CAs, the resulting complexes have a much larger molecular mass resulting in a slower rotation in water and a contrast enhancement. CDs have proved their interest as catalyst, drug delivery system, ligands, constituents of chiral separation media, monolayers, and particularly, as host molecules forming inclusion complexes. The size and shape of the cavity of CDs play an essential role in the formation of these inclusion complexes, with hydrophobic interactions, H-bonding and Van der Waals forces. CDs are widely used in supramolecular chemistry due to their ability to bind hydrophobic molecules within their cavity when they are dissolved in polar solvents such as water. Functionalized CDs have found applications as molecular reactors, enzyme mimics (catalysts), molecular machines, and electrode surface modifiers. They are among the most promising and widely employed oligosaccharide hosts for drug complexation. Indeed, CD-encapsulated drugs usually have good water solubility, a better bioavailability, a longer half-life under physiological conditions, unhindered excretion and no extra toxicity. Therefore cyclodextrins appear as promising carriers for the in vivo vehiculation of Gd(III) complexes. For instance, the following contrast agents comprising a CD have been disclosed by Nocera's team in 1996 (Mortellaro et al., J. Am. Chem. Soc. 1996, 118, 7414-7415) and Aime's team in 2000 (Skinner et al., J. Chem. Soc., Perkin Trans. 2 2000, 1329-1338):

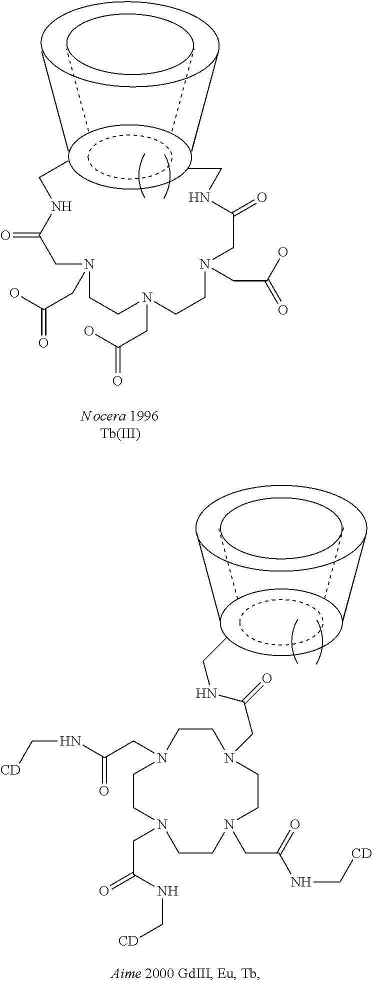

However, the CD-complex can not penetrate the lipophilic barriers of membrane cells; it carries the drug and enhances its concentration at the membrane surface. It facilitates the drug absorption after dissociation.

Previous work has established that concomitant administration of Gd and CD, linked by covalent bond or non-covalent interactions can increase τᵣ and consequently the water proton relaxation rates (r₁). Thus, a relaxivity more than five times higher, than those reported for the systems commonly used in clinical practice, was reached. Indeed, relaxivities of the order of r₁=25 mM⁻¹s⁻¹ were reported with systems with molecular weight slightly higher than 4 kDa.

Complexes clinically available are coordinated with nine ligands, the polyaminopolycarboxylate ligand providing eight donors and a water molecule occupying the ninth coordination site.

The enhancement of relaxivity occurs by two mechanisms: (i) reduction of the number of coordinated water molecules, and so the contribution of inner-sphere water (directly coordinated to Gd(III)); (ii) increase of the relaxation issue of the second-sphere water (H-bonded to lone pairs on the carboxylate oxygen atoms). It is suggested that the high density of hydroxyl groups on the crowns of the CD cavities may yield to strong interactions with the water molecules on the surface of the complexes, and it lengthens their lifetime in the proximity of the paramagnetic centre. The hydrogen-bond network involving the coordinated water molecule(s) will be reinforced. On the other hand, such tight arrangement appears responsible for an enhanced contribution to the observed relaxivity arising from water molecules in the second coordination sphere of the metal centre. Therefore, these supramolecular complexes bring about a beneficial effect on the exchange process of coordinated water molecules and they are very likely to provide a significant improvement in terms of sensitivity of the MRI.

About Smart Probes:

CAs for specific targets were recently reported in the literature with the aim to recognize a given disease. Aime has recently developed a smart CA responsive to the concentration of free thiols in tissues in vitro (Carrera et al., Dalton Trans. 2007). A few smart CAs were described for in vivo applications. For example, an enzymatic activity was observed by converting an MRI-inactivated agent to an activated MRI agent by using Gd-complex containing beta-galactopyranose (Chang et al., Bioconjugate 2007). McIntyre's group has also detected the activity of proteinase based on the concept of a solubility switch from hydrophilic to hydrophobic that significantly modifies the pharmacokinetic properties of the agent (Lepage et al., Molecular Imaging 2007).

About the Biological Targets:

There is still a need for identifying and quantifying extracellular targets involved in challenging pathologies. Observing these targets enables to assess associated dysfunctions, but also to evaluate the efficiency of putative therapeutic agents in vivo. Biological targets can be any extracellular protein displaying an enzymatic cleavage activity. Said biological target can be a membrane-bound enzyme or a secreted enzyme. Indeed, the extracellular compartment contains a variety of enzymes involved in the remodelling of the extracellular matrix, in the digestion of nutrients, etc. The growing interest towards the aforementioned extracellular membrane targets clearly illustrates the need for an efficient way to image them by a simple and reliable method to assess their activity in vivo.

In conclusion, there is a need of more efficient new contrast agents for MRI imaging, especially providing for a higher relaxivity (r1), which would not have the detrimental side effects (such as the triggering of an allergic response) of other contrast agents based on gadolinium. Moreover, elaboration of a smart contrast agent, which could provide a visualisation and possibly also a quantification of a specific activity in vivo, constitutes a real challenge in the field of molecular imaging.

### SUMMARY OF THE INVENTION

After years of studies, the Applicant has discovered new contrast agents for MRI imaging.

The contrast agent according to the invention can be a compound comprising:
one cyclodextrin, whose truncated-cone-shaped structure defines a central axis, a first and a second openings along said axis,
one paramagnetic element, located on said cyclodextrin axis, outside said structure and proximate to the first opening,
one or several coordination ligand(s) of the paramagnetic element which coordinate(s) said paramagnetic element,
one arm of formula

-A

covalently bound to the cyclodextrin, proximate to the second opening,
wherein A is a carbon group able to form an inclusion complex with the cyclodextrin.

In the context of the present text, “one” means “at least one”.

Further, the Applicant proposes new bioactivable contrast agents for MRI imaging. The bioactivable contrast agent according to the invention can be a compound comprising:
one cyclodextrin, whose truncated-cone-shaped structure defines a central axis, a first and a second openings along said axis,
one paramagnetic element, located on said cyclodextrin axis, outside said structure and proximate to the first opening,
one or several coordination ligand(s) of the paramagnetic element which coordinate(s) said paramagnetic element,
one mobile arm of formula

-A-X

covalently bound to the cyclodextrin, proximate to the second opening,
wherein X is a cleavable biometabolizable moiety,
A is a spacer between the cyclodextrin and X, wherein A is a carbon group able to form an inclusion complex with the cyclodextrin.

The contrast agents according to this invention enable to detect in vitro and vivo extracellular biological processes which translate into the modulation of the IRM signal of these agents. Thus, they provide for an accurate, specific and reliable diagnosis, which will result in an improvement in the treatment and in the survival rate of patients. These contrast agents also allow for a non-invasive monitoring of these pathological processes (such as cardiovascular diseases, including myocardial infarction, and cancers) with time and following treatment. Moreover, the bioactivable contrast agents of this invention make possible not only to visualize, but also to quantify, the biochemical processes involved, contrary to the contrast agents of the prior art which accumulate in the tissues without being modified biochemically.

### DETAILED DESCRIPTION

### Description of the CD

Cyclodextrins (sometimes called cycloamyloses) make up a family of cyclic oligosaccharides, composed of 5 or more α-D-glucopyranoside units linked 1→4. Typical cyclodextrins contain a number of glucose monomers ranging from six to eight units in a ring, creating a truncated-cone shape (see scheme 1). This structure is defining a central axis (1) and two openings (a first (2) and a second (3) opening) along said axis (1).

Preferably, the MRI contrast agent comprises one cyclodextrin selected from the group consisting of alpha-cyclodextrin (six membered sugar ring molecule), beta-cyclodextrin (seven membered sugar ring molecule) and gamma-cyclodextrin (eight membered sugar ring molecule).

More preferably, the MRI contrast agent comprises at least one beta-cyclodextrin.

The cyclodextrin may be functionalized, in particular by lipophilic groups, in order to improve their bioavailability, for instance.

In the present description, the term “cyclodextrin” will thus designate alpha-, beta- or gamma-cyclodextrin, which is functionalized or not.

Description of the Paramagnetic Element:

The paramagnetic feature of an element is the result of the presence of unpaired electrons.

Preferably, the MRI contrast agent comprises at least one paramagnetic element selected in the group of lanthanides such as gadolinium and dysprosium; transition metals such as manganese, iron, cobalt, and chromium; and magnesium. More preferably, the paramagnetic element of the invention is gadolinium.

According to the invention, the paramagnetic element is located along the axis of the cyclodextrin, and proximate to the first opening. The first opening is preferably the smallest opening of the cyclodextrin.

Description of the Coordination Ligand:

The choice of the coordinating group affects the number and residence lifetime of water molecules being in the proximity of the paramagnetic centre and therefore the relaxivity. The one or several coordination ligand(s) of the paramagnetic element coordinate(s) said paramagnetic element and is (are) covalently bound to the cyclodextrin, proximate to the first opening of the cyclodextrin.

If there are several ligands, they may be the same or different. Preferably, they are the same.

The coordination ligand can be monodentate, bidentate, tridentate, quadridentate, or with five or more coordination sites.

If the coordination ligand is monodentate, it can correspond to formula I

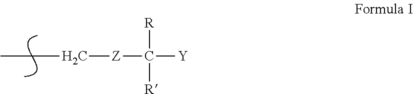

wherein R and R′ are independently hydrogen or an alkyl chain,
Z is NR or O,
Y is CO₂, PO₃²⁻ or SO₃²⁻.

A preferred monodentate ligand has the following formula: —CH₂—O—CH₂—COO⁻. Preferably, from 5 to 7 monodentate ligands are grafted to the cyclodextrin.

If the coordination ligand is bidentate, it can correspond to one of formulas II to VIII:

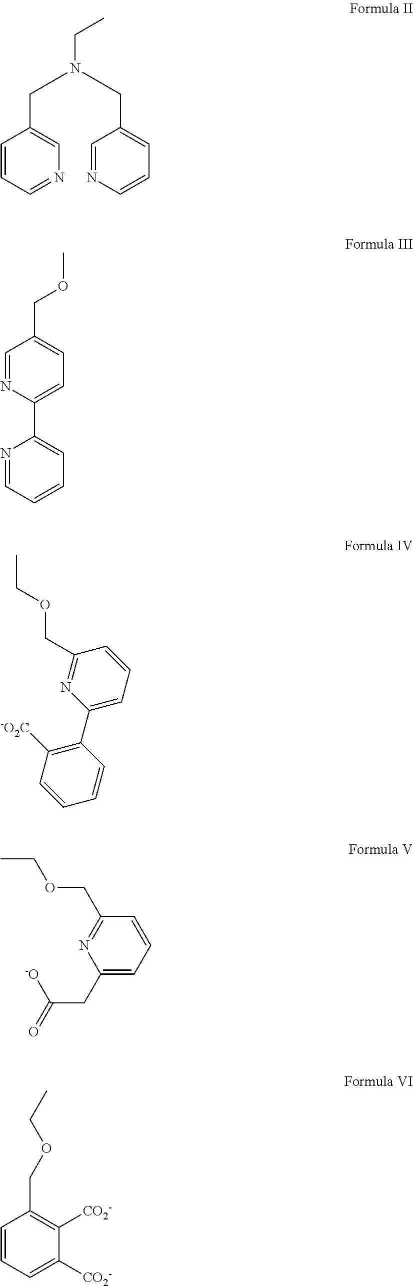

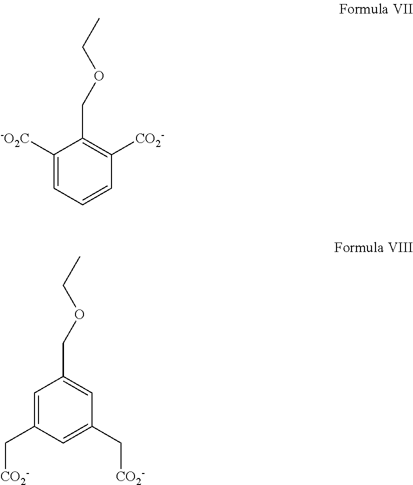

The cyclodextrin may bear from three to five bidentate ligands, for instance.

If the coordination ligand is tridentate, it can correspond to one of formulas IX to XV:

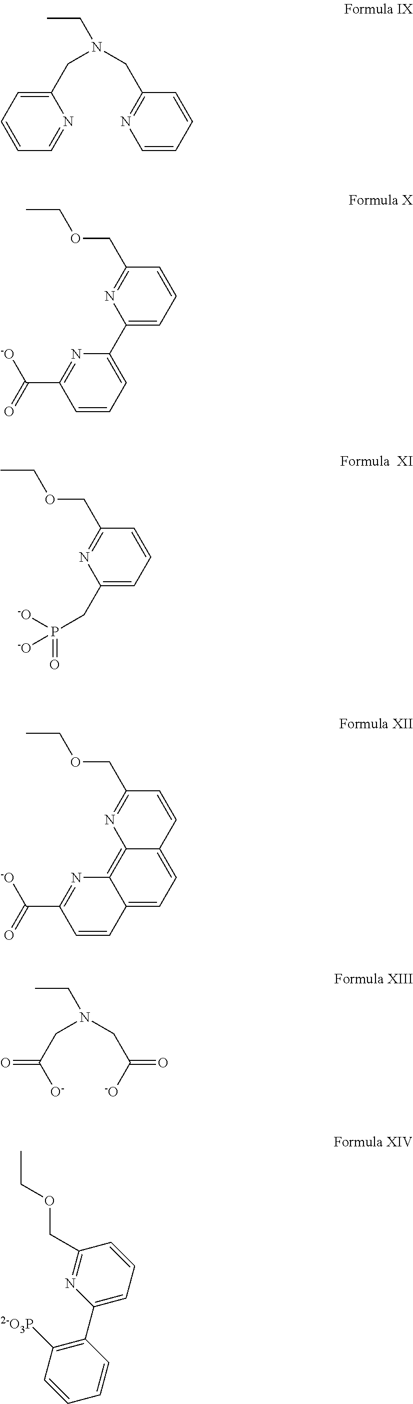

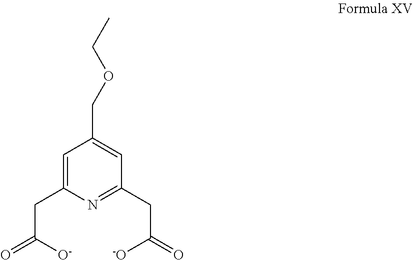

The cyclodextrin may bear two tridentate ligands, for instance.

If the coordination ligand is quadridentate, it can correspond to one of formulas XVI to XXIII:

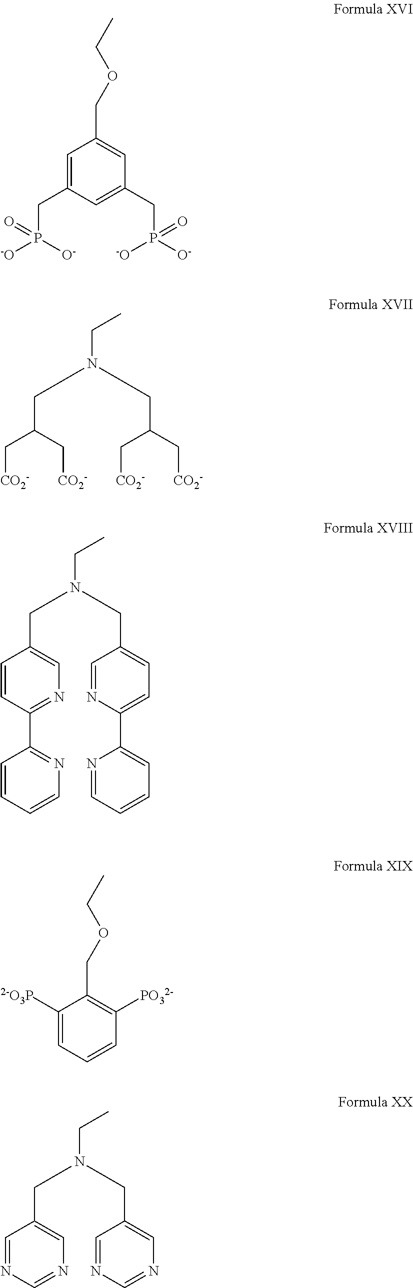

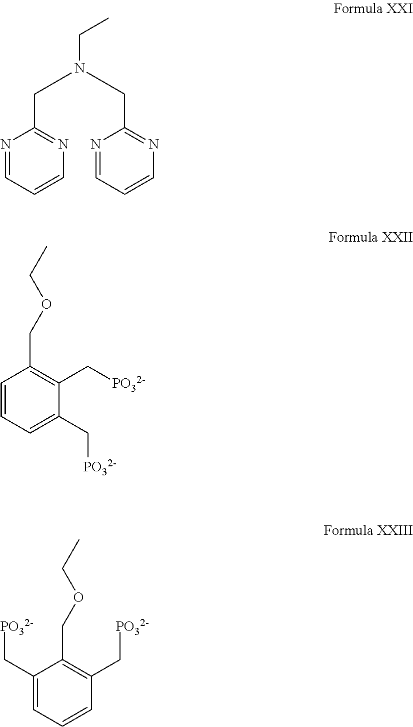

In a preferred embodiment, the ligands are seven monodentate ligands, preferably having formula: —CH₂—O—CH₂—COO⁻.

In another preferred embodiment, the ligands are four bidentate ligands, preferably having formula V.

In another preferred embodiment, the ligands are two tridentate ligands, preferably having formula XI.

Description of the Arm:

The MRI contrast agent according to the invention comprises an arm A, which is a carbon group.

Preferably, A can be an alkyl group, an aryl group, or an aryl-alkyl group, comprising preferably from 8 to carbon atoms, optionally comprising one or several heteroatoms, unsubstituted or substituted by alkyl, aryl, carboxylate, phosphonate, sulphonate or other functions.

The arm according to the invention advantageously comprises a phenyl ring.

According to a first embodiment, the contrast agent comprises an arm -A of one of formulas XXIV to XXVII:

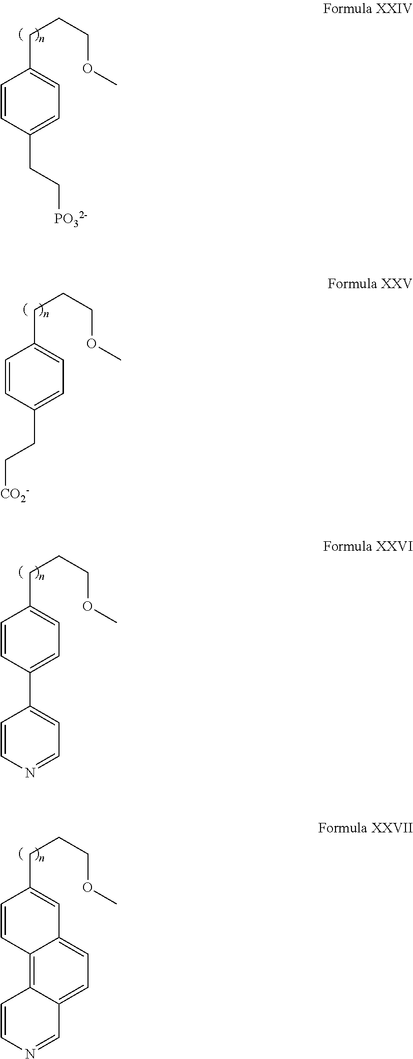

wherein n is 1 to 10.

In some embodiments, a terminal functional group of the arm A, for instance carboxylate or phosphonate, can be able to coordinate the paramagnetic element.

According to a second embodiment of the invention, the bioactivable contrast agent for MRI imaging comprises an X moiety, which is grafted on A, which acts as a spacer. The spacer A, which is preferably mobile, can be preferably an alkylene chain, an arylene chain, or an aryl-alkylene chain, comprising preferably from 8 to 20 carbon atoms, optionally comprising one or several heteroatoms, unsubstituted or substituted by alkyl, aryl, carboxylate, phosphonate, sulphonate or other functions.

The X moiety is a cleavable biometabolizable moiety. The term “biometabolizable” means that X is a substrate of a biological target (an extracellular enzyme, for instance). When the target is peptidase or a protease, X preferably comprises a peptidic sequence, more preferably a peptidic sequence that is specifically recognized by the target enzyme in vivo.

Interesting targets for molecular imaging are proteases or peptidases, i.e. enzymes which specifically cleave a peptide bond of proteins or peptides displaying a given amino acid sequence. Also covered are osidases, which cleave specific osidic bonds (in this case, X can be a sugar-containing molecule). Also covered are esterases, which cleave ester bonds or phosphonate bonds or amide bond (hydrolase) (in this case, X will be an ester-, phosphonate- or amide-containing molecule, respectively). Examples of specific enzyme which may be targeted according to this invention are the angiotensin I converting enzyme and matrix metalloproteinases (MMPs).

Use of the Contrast Agent:

The Applicant provides an excellent contrast agent for MRI imaging.

The present invention also relates to the method of acquiring an image, comprising a) the administration of the contrast agent according to the invention to a tissue, cell or patient, and b) the acquisition of a magnetic resonance image of said cell, tissue or patient. Preferably, the MRI contrast agent can be administrated in a composition containing a pharmaceutically acceptable carrier. According to an embodiment of this invention, the composition, and preferably the contrast agent itself, may include at least one drug which can be detected by IRM. Due to its excellent inclusion capacity and to its large cavity, the beta-CD may act as a vector for this drug. Moreover, the high molecular weight structure (M=6 kDa) of this cyclodextrin can increase τᵣ and consequently, the water proton relaxation rates.

Thanks to its better efficiency, the amount of the contrast agent and the stronger coordination of the paramagnetic element by the selected ligands reduce the risk of intoxication and of allergic reaction for the patient.

According to an embodiment of this invention, the MRI contrast agent may be used to detect ligands which are found in excess in blood due to a biochemical reaction in relation to a specific disease. These ligands may be detected using this contrast agent by intermolecular complexation of the paramagnetic element inducing a variation in the coordination of this element and in the degree of hydration, as illustrated on Scheme 2. In this situation, the contrast agent thus emits a first signal corresponding to the binding of the arm of the contrast agent with the biological target and a second signal corresponding to the coordination of the biological ligand.

The bioactivable contrast agent according to the invention constitutes also a new and efficient contrast agent for MRI imaging. Therefore, the present invention also relates to the method of acquiring an image, comprising a) the administration of the bioactivable contrast agent according to the invention to a tissue, cell or patient, and b) the acquisition of a magnetic resonance image of said cell, tissue or patient. Preferably, the bioactivable contrast agent can be administrated in a composition including a pharmaceutically acceptable carrier. The recorded signal, triggered by the binding of the arm of the contrast agent with the biological target, will be designated as “Signal 1”.

The biochemical reaction on the cell membrane target preferably cleaves the arm at the biometabolizable moiety. The carbon group, or spacer, which remains after biochemical cleavage can then form an inclusion complex with the CD. Regarding the MRI aspects, the influence of the inclusion complex is significant on the relaxivity of water proton linked to the paramagnetic element. The biochemical reaction brings about a mass reduction and possibly induces an inclusion phenomenon which disturbs the relaxivity and then the MRI signal. Therefore, the recorded signal after the cleaving of the biometabolizable moiety is different from “Signal 1” and will be designated as “Signal 2” (see scheme 3).

The signals emitted by the contrast agents of this invention may be detected by any method known to the person skilled in the art, for instance as relaxivity, frequency or phase variations.

In another preferred embodiment, said cleavable biometabolizable moiety comprises a moiety which can be detected by any other imaging technique than MRI, for instance nuclear imaging (tomography, PET, SPECT . . . ), optical detection (fluorescence spectroscopy techniques . . . ), or ultrasound, as a further signal. According to this embodiment, MRI contrast agents of the invention can thus become multi-modal probes, which allow a multi-modal in vivo detection of biological phenomena.

Synthesis:

The new MRI contrast agent according to the invention and the bioactivable contrast agents according to the invention can be prepared by two grafting steps on each face of the CD. The synthesis strategy needs known and selective succession of protection and deprotection steps. In any cases, the paramagnetic element may be introduced at the last step.

The following synthesis strategies are examples of preparation of contrast agents according to the claims. These syntheses are not limitative examples.

The synthesis strategy of the new contrast agents according to the invention can comprise two parts:
(i) the synthesis of the CD with ligand(s);
(ii) the functionalization of the CD with the (optionally mobile) arm.

Further, the synthesis of the bioactivable contrast agent according to the invention can comprise two further parts:
(iii) the synthesis of the biometabolizable moiety;
(iv) the grafting of the biometabolizable moiety onto the functionalized CD.

Parts (i) and (iii) can start independently.

(i) Synthesis of the CD with Ligand(s)

The ligands will be synthesized in one or several steps from commercially available products, by any synthesis strategy known by the skilled person.

The ligand(s) may be grafted onto the CD for example by an ether function, which is a biologically stable linker. After selective protections of the lower and upper faces of the CD, the grafting of the ligand(s) can be envisaged on the de-protected primary alcohols by adding a base and a precursor of the ligand(s) (Tian et al., J. Org. Chem. 2000).

For instance, the tridentate ligand of Formula XI may be grafted to the cyclodextrin according to a method including: the perbenzylation of the cyclodextrin by addition of benzyl chloride in the presence of pyridine. Positions A and D are then deprotected with DIBAL as described by Pierce and Sinaÿ, (Angew. Chem. Int. Ed., 2000, 39, 3610-3612). Separately, the ligands are prepared by the Arbusov reaction (Med. Chem. Lett., 2007, 17, 1466-1470) conducted on the commercial 2-bromomethyl-6-methylpyridine, followed by radical bromation. These ligands are then grafted in the presence of a strong base onto the A and D free positions. Both phosphonates are then deprotected and debenzylation is conducted. This method is partly illustrated on Scheme 4.

The bidendate ligand of formula V can be synthesized in three steps from the commercially available 6-methylpicolinic acid (see Scheme 5) (Ijiun et al., Bioorg. Med. Chem. 2006). The precursor can be esterified by methanol in acidic medium. The radical bromination of the methyl group will lead to the corresponding 6-bromomethylpicolinic methylester. The grafting can be made according to the synthesis strategy of Scheme 6. Specifically, after deprotecting the four primary alcohols on positions A to D with DIBAL, as described above with reference to Scheme 4, the grafting of the four bidentate ligands is made in the presence of a base such as NaH, by adding 6-bromomethylpicolinic methylester prepared as described above. The benzyl alcohols and the carboxylic functions on the pyridin units are then deprotected.

The monodentate ligand —CH₂—O—CH₂—COO⁻ may be grafted onto the cyclodextrin by adding sodium iodoacetate thereto, in a solvent such as pyridine.

(ii) Functionalization of the CD with the Arm

Hydroxyl groups present at the C2-, C3-, and C6-positions compete for the reagent and make selective modification extremely difficult. Of the three types of hydroxyl groups present, those at the C6-position which correspond to primary alcohol are the most basic (pKa 15-16), those at the C2-position are the most acidic (pKa 12.1), and those at the C3-position are the most inaccessible and not easily available for further modifications. The secondary side is more hindered than the primary side due to the presence of twice the number of hydroxyl groups. Hydrogen bonding between hydroxyl groups at C2- and C3-positions makes them rigid and less flexible as compared to C-6 ones. All these factors make the secondary side less reactive and harder to selectively functionalize than the primary face.

Recently, the direct monosubstitution at C2-position of unprotected beta-CD by treatment with sodium ethoxide in DMSO has been published (Masurier et al., Eur. J. Med. Chem. 2005, 40, 615-623, and Masurier et al., Carbohydrate Res. 2006, 341, 935-940) (Scheme 7). The degree of functionalization was controlled by the amount of alkali. This methodology can be applied in this case, with the moiety A of the mobile arm.

The moiety A of the mobile arm is synthesized in one or several steps from the commercially available products, by any synthesis strategy known by a skilled person.

For example, the 2-bromoethylbenzylamine is synthesized from the commercial 1,4-dihydro-3(2H)isoquinolinone (Scheme 8) (Ikeda et al., Tetrahedron, 1977, 33, 489-495). After hydrolysis of the cyclic amide in acidic medium, the carboxylic acid function is reduced and the corresponding alcohol substituted by a bromine atom. A protection step of amine function can also be considered. Others structures (various positions of methylamine group, aromatic substitutions) could be also considered to optimize the inclusion complex formation. The structures of these new functionalized CDs can be confirmed by NMR, X-Ray and Mass Spectrometry analysis.

As a general rule, the arm may be obtained from commercial derivatives of para-dibromobenzene, by adding oxiranne thereto (Bernstein et al., Med. Chem., 1986, 29, 2477-2483), so as to obtain an alcohol which is then reacted with hydrobromic acid.

(iii) Synthesis of the Biometabolizable Moiety

As described above, the biometabolizable moiety can be any cleavage substrate, specific of a given enzyme. It can be a protein or peptide, an ose, or any other group with an ester function. The skilled person in the art will be able to choose a specific biometabolizable moiety according to the biological target which he wishes to identify and/or quantify. It will be synthesized in one or several steps from commercially available products, by any synthesis strategy known by the skilled person.

If the biometabolizable moiety comprises a moiety which can be detected by any other imaging technique than MRI, in order to provide a multi-modal probe, the synthesis strategy will be adapted by the skilled person.

(iv) Grafting of the Biometabolizable Moiety on the Functionalized CD

The biometabolizable moiety will be linked to the mobile arm on the CD by any available technique, for instance a peptide linker.

The following example is given for illustration purpose only and is not intended to restrict the scope of this invention, which is defined by the attached claims.

### EXAMPLES

### Example 1

### Synthesis of a Contrast Agent with Bidentate Ligands

Step 1. In a 500 mL round bottom flask, 10 g of β-cyclodextrin (8.81 mmol) were dissolved in 100 mL of distilled pyridine. With stirring and under inert atmosphere, 10.3 g of TBDMSCl (tertiobutyl-dimethyl-silyl chloride (68.4 mmol), previously dissolved in 100 mL of distilled pyridine, were added in the round bottom flask. The temperature of the reaction mixture was lowered to 0° C. during 3 hours. Then, the reaction mixture was left at room temperature during 24 hours. The product then precipitated by adding water. The powder obtained was purified by silica gel chromatography, wherein the eluent was a chloroform/methanol/water mixture (40/10/1; v/v/v).

The reaction yield of this step was 58%.

Step 2. In a 500 mL round-bottom flask with stirring and under inert atmosphere, 2 g of Step 1-product were dissolved in 50 mL of distilled pyridine. 20 mL of benzoyl chloride were added. The reaction mixture was heated at 100° C. during 48 hours. The mixture was reduced by an half, and then placed in an ice bath. 60 mL of methanol were added dropwise. The mixture was then evaporated to obtain a syrup, in which 105 mL of methanol and 30 mL of water were added. The precipitate obtained was filtered and dried. The powder obtained was purified by silica gel chromatography, wherein the eluent was a cyclohexane/acetone mixture (3/2; v/v).

The reaction yield of this step was 57%.

Step 3. In a 100 mL round-bottom flask, 2 g of Step 2-product were dissolved in 30 mL of methylene chloride. 2 mL of distilled boron trifluoride (15.8 mmol) were added. The reaction mixture was then stirred at room temperature during 72 hours. The reaction mixture was then diluted with 30 mL of methylene chloride, then poured into 100 mL of water/ice mixture (1/1; v/m). The organic phase was washed with 25 mL of NaHCO₃ solution, then with 25 mL of water, and dried with MgSO₄. After filtration, the solvent was removed.

The reaction yield of this step was 78%.

Step 4. 1 g of 2-cyano-6-methylpyridine (8.75 mmol) was introduced in a 20 mL round-bottom flask. 10 mL of 6M hydrochloric acid were added. The reaction mixture was refluxed during 24 hours. 130 mL of acetonitrile were added. The precipitate was filtered and the solvent was evaporated.

The reaction yield of this step was 94%.

Step 5. In a two-neck round bottom flask, 2 g of Step 4-product were dissolved in 10 mL of methanol. 3 ml of 96% sulphuric acid were added dropwise from a dropping funnel to the flask. The reaction mixture was refluxed during 24 hours, then cooled at room temperature, poured into 30 mL of ice water and neutralized with a Na₂CO₃ solution. The product was extracted with several portions of methylene chloride. The solvent was then evaporated.

The reaction yield of this step was 74%.

Step 6. 1.1 g of Step 5-product (7.28 mmol) were introduced in a 250 mL round-bottom flask. 1.3 g of NBS (N-bromo-succidimine, 7.3 mmol), 80 mL of CCl4 and a small quantity of benzoyl peroxide were added. The reaction mixture was refluxed during 24 hours. The solid obtained was filtered and drawn off. The solvent was evaporated.

The product obtained was purified on a silica gel chromatography (eluent: methylene chloride).

The reaction yield of this step was 32%.

Step 7. The coupling of Step 3-product and Step 6-product can be made in a two-solvent mixture (pyridine and 2,6-lutidine) with a reflux. By addition of trichlorogadolinium hexahydrate, the contrast agent of formula XXVIII is obtained.

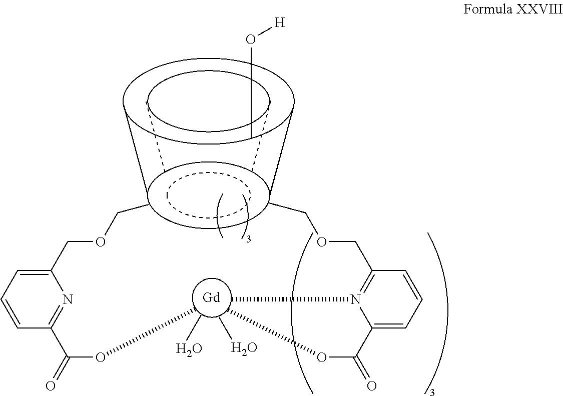

Based on the obtained contrast agent of formula XXVIII, Part (ii) of the general synthesis is implemented to produce a contrast agent according to the invention.

### Example 2

### Synthesis of a Contrast Agent with Monodentate Ligands

A beta-cyclodextrin bearing acetate ligands is synthetized. The functionalized cylodextrin thus obtained is grafted with a spacer bearing a biometabolizable group and then complexed with trichlorogadolinium hexahydrate, to afford a contrast agent according to this invention.

### Step 1: Synthesis of 2,3-dimethyl-β-cyclodextrin 1b

In a 500 mL round bottom flask, 10 g of β-cyclodextrin (8.81 mmol) were dissolved in 100 mL of distilled pyridine. With stirring and under inert atmosphere, 10.3 g of TBDMSCl (tertiobutyl-dimethyl-silyl chloride (68.4 mmol), previously dissolved in 100 mL of distilled pyridine, were added in the round bottom flask. The temperature of the reaction mixture was lowered to 0° C. during 3 hours. Then, the reaction mixture was left at room temperature during 24 hours. The product then precipitated by adding water. The powder obtained was purified by silica gel chromatography, wherein the eluent was a chloroform/methanol/water mixture (40/10/1; v/v/v). The reaction yield of this step was 58%.

The 6-persilylated-beta-cyclodextrin (0.01 mol, 20 g) thus obtained was dissolved in dry THF (200 mL), then NaH was added (0.26 mol, 10.4 g, dispersed in oil 60%). Iodomethane (0.57 mol, 28.5 mL) was added by small portions for 1 h. After 17 h the excess of sodium hydride was neutralized by addition of methanol (50 mL). Solvents were removed by evaporation under reduced pressure. The residue was suspended in cyclohexane (300 mL). The solid was filtered and the solvent were evaporated under pressure. The solid (0.589 mmol, 2 g) was dissolved in methanol (15 mL) and ammonium fluoride (14 mmol, 0.046 g) was added. The solution was refluxed for 5 hours. The reaction was monitored by TCL (CHCl₃/MeOH/H₂O 40:10:1). Methanol was removed by evaporation. The crude product was dissolved in CH₂Cl₂ (60 mL) and poured into water (200 ml). The organic layer was separated and dried over MgSO₄ and removed by evaporation under pressure. The product was purified by column chromatography on silica gel eluting with CH₂Cl₂ then CHCl₃/MeOH/H₂O (40:10:1). A white powder 1b was obtained (77.8%). The corresponding compound is illustrated on Scheme 9, wherein R1=CH₃ and n=3-7.

### Step 2: Grafting of the Ligands

### Step 2a: Synthesis of 7-(6-O-acetate)-β-cyclodextrin 2a

Dry β-cyclodextrin 1a (2.2 mmol, 2.5 g) was diluted with anhydrous DMF (100 ml) and degassed thoroughly. Anhydrous pyridine was added via a syringe (30 mmol, 2.3 g, 2.35 mL) followed by the addition of sodium iodoacetate (17 mmol, 3.53 g). The mixture was stirred at 90° C. for 72 h. The solvent was evaporated under pressure and the sticky brownish solid was washed with acetone (250 ml). The insoluble solid was filtrated on a glass frit. The yellowish brown solid was collected and filtrated on silica gel with a mixture CH₂Cl₂/MeOH (1/1) as eluent. 2a was obtained after evaporation under pressure.

### Step 2b: Synthesis of 7-(6-O-acetate)-2,3-dimethyl-β-cyclodextrin 2b

Dry 2,3-dimethyl-β-cyclodextrin 1b (0.0751 mmol, 100 mg) was diluted with anhydrous DMF (7 ml) and degassed thoroughly. Anhydrous pyridine was added via a syringe (1.02 mmol, 0.084 ml) followed by the addition of sodium iodoacetate (2.63 mmol, 550 mg). The mixture was stirred at 90° C. for 72 h. The solvent was evaporated under pressure and the sticky brownish solid was washed with acetone (250 ml). The insoluble solid was filtrated on a glass frit. The filtrate was evaporated under pressure and filtrated on silica gel with a mixture CH₂Cl₂/MeOH (1/1) as eluent. 2b was obtained after evaporation under pressure.

### Step 3: Synthesis of the Spacer

This step is illustrated on Scheme 10.

Under argon atmosphere, to a stirred solution of p-dibromobenzene (4.2 mmol, 1 g) in THF (20 mL) was added n-BuLi (9.3 mmol, 6.2 mL, 1.5 M in hexane) at −78° C. The reaction mixture was stirred at −78° C. for 30 min. The oxiranne (12.7 mmol, 0.56 g) and BF₃.Et₂O (12.7 mmol, 1.8 g) were added at −78° C. and stirred for 5 hours at the same temperature. After quenching with diluted H₂SO₄, the reaction mixture was extracted with Et₂O (50 mL×3), and the combined organic phase was dried over anhydrous MgSO₄. The solvents were removed under vacuum and the crude product was purified by silica gel column chromatography (ethyl acetate/cyclohexane 1:10 v/v). The diol was obtained with 49% yield.

A suspension of 1.7 g of the diol in 25 mL of 48% hydrobromic acid was refluxed and stirred for 15 h. The mixture was extracted with methylene chloride and the extracts were dried, filtrated and concentrated under vacuum to yield a brown solid. Recristallization from cyclohexane afforded 2.7 g (88%) of p-di(bromoethyl)benzene as pale yellow needle.

Triethylphosphite (13 mmol, 3.5 mL) was added slowly to the p-di(bromoethyl)benzene (40 mmol, 7 g) in dry toluene. The mixture was stirred for 42 h at reflux under nitrogen. Toluene was removed under vacuum and methanol (40 mL) was then added. The precipitate was collected and washed with methanol (10 mL). The filtrate was evaporated and the resulting oil (4.2 g) was purified by distillation under vacuum. The product was obtained as a colorless liquid (3.15 g, 91%).

### Step 4: Grafting of the Spacer on the Cyclodextrin

A spacer or arm is grafted onto cyclodextrin 2a according to the following procedure, as illustrated on Scheme 11. It can similarly be grafted onto cyclodextrin 2b.

2a (0.88 mmol, 1 g) is dried for 48 h under reduced pressure at 120° C. and dissolved in 40 mL of dry DMSO. Under nitrogen 5 mL of EtONa solution (200 mg of Na in 50 mL of EtOH) is added to 2a and stirred for 14 h. The electrophile reagent (0.88 mmol, 0.307 g) in 5 mL of solvent is added dropwise to the reaction medium. The mixture is stirred for 9 h. Acetone (500 mL) is added to precipitate the crude product 3a. After filtration, the solid residue is purified by chromatography on silica gel (12:7:4, EtOAc-isopropanol-water v/v/v).

### Step 5: Complexation of the Gadolinium

a) Method A

Dry compound 3a is dissolved in ultrapure water and gadolinium chloride hexahydrate is added. The solution is stirred and the pH is adjusted to a pH 8-9 using aqueous sodium bicarbonate solution (1 mol·L⁻¹). The solution is centrifuged, and gadolinium residual is filtered.

b) Method B

Dry compound 3a is dissolved in ultrapure water /NaCl 0.9% w/v, and gadolinium chloride hexahydrate is added. The solution is stirred and the pH is adjusted to a pH 6.9-7.4 using aqueous NaOH solution (1 mol·L⁻¹).

c) Method C

Dry compound 3a is dissolved in ultrapure H₂O. The pH of the solution is adjusted to 6.5 with aqueous NaOH solution (1 mol·L⁻¹). Gadolinium trichloride hexahydrate is dissolved in water and the pH is also adjusted to 6.5 with aqueous NaOH solution (1 mol·L⁻¹). The Gd (III) solution is added to the cyclodextrin solution and the pH is stabilized between 5.5 and 6.0 with aqueous NaOH solution (1 mol·L⁻¹). The mixture solution is stirred at room temperature and the pH is then adjusted to 8.0.

### Example 3

### Use of the Contrast Agents of this Invention

The contrast agents synthetized as described in Examples 1 and 2 above may be included in a composition intended to be administered to a subject. Once the specific enzymes targeted have cut the bond between the biometabolizable group and the spacer in the arm, a signal is emitted by the smart probe as a result of the inclusion of the spacer within the cyclodextrin. This signal may be compared to that emitted before the action of these enzymes, so as to provide for a quantification of the enzymatic activity.

1-13. (canceled)

14. A contrast agent for MRI imaging comprising:
one cyclodextrin, whose truncated-cone-shaped structure defines a central axis, a first and a second openings along said axis,
one paramagnetic element, located on said cyclodextrin axis, outside said structure and proximate to the first opening,
one or several coordination ligand(s) of the paramagnetic element which coordinate(s) said paramagnetic element,
one arm of formula
-A
covalently bound to the cyclodextrin, proximate to the second opening,
wherein A is a carbon group able to form an inclusion complex with the cyclodextrin.

15. A bioactivable contrast agent for MRI imaging comprising:
one cyclodextrin, whose truncated-cone-shaped structure defines a central axis, a first and a second openings along said axis,
one paramagnetic element, located on said cyclodextrin axis, outside said structure and proximate to the first opening,
one or several coordination ligand(s) of the paramagnetic element which coordinate(s) said paramagnetic element,
one mobile arm of formula
-A-X
covalently bound to the cyclodextrin, proximate to the second opening,
wherein X is a cleavable biometabolizable moiety,
A is a spacer between the cyclodextrin and X, wherein A is a carbon group able to form an inclusion complex with the cyclodextrin.

16. The MRI contrast agent according to claim 14, wherein the cyclodextrin is selected in the group consisting of alpha-cyclodextrin, beta-cyclodextrin and gamma-cyclodextrin.

17. The MRI contrast agent according to claim 16, wherein the cyclodextrin is a beta-cyclodextrin.

18. The MRI contrast agent according to claim 14, wherein the paramagnetic element is selected from the group consisting of lanthanides such as gadolinium and dysprosium; transition metals such as manganese, iron, cobalt, and chromium; and magnesium.

19. The MRI contrast agent according to claim 18, wherein the paramagnetic element is gadolinium.

20. The MRI contrast agent according to claim 14, wherein the first opening is the smallest opening of the cyclodextrin.

21. The MRI contrast agent according to claim 14, wherein the coordination ligand(s) correspond(s) to formula V:

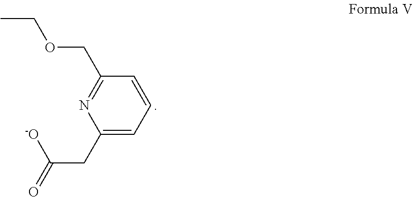

22. The MRI contrast agent according to claim 14, wherein the coordination ligand(s) correspond(s) to formula XI:

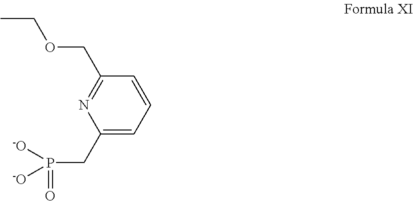

23. The MRI contrast agent according to claim 14, wherein the coordination ligand(s) correspond(s) to formula I: 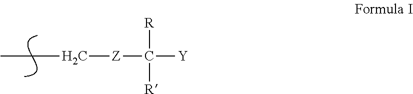
wherein R and R′ are independently hydrogen or an alkyl chain,
Z is NR or O,
Y is CO₂⁻, PO₃²⁻ or SO₃²⁻.

24. The MRI contrast agent according to claim 15, wherein the cleavable biometabolizable moiety X comprises a peptidic sequence.

25. Method of acquiring an image, comprising:
a) the administration of the contrast agent according to claim 14 to a tissue, cell or patient, and
b) the acquisition of a magnetic resonance image of said cell, tissue or patient.

26. The method according to claim 25, comprising administering a composition comprising said contrast agent and a pharmaceutically acceptable carrier.

27. The MRI contrast agent according to claim 15, wherein the cyclodextrin is selected in the group consisting of alpha-cyclodextrin, beta-cyclodextrin and gamma-cyclodextrin.

28. The MRI contrast agent according to claim 27, wherein the cyclodextrin is a beta-cyclodextrin.

29. The MRI contrast agent according to claim 15, wherein the paramagnetic element is selected from the group consisting of lanthanides such as gadolinium and dysprosium; transition metals such as manganese, iron, cobalt, and chromium; and magnesium.

30. The MRI contrast agent according to claim 29, wherein the paramagnetic element is gadolinium.

31. The MRI contrast agent according to claim 15, wherein the first opening is the smallest opening of the cyclodextrin.

32. The MRI contrast agent according to claim 15, wherein the coordination ligand(s) correspond(s) to formula V:

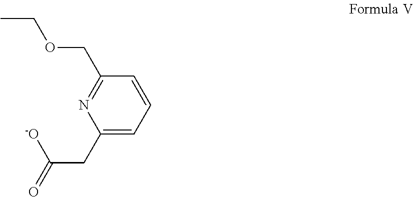

33. The MRI contrast agent according to claim 15, wherein the coordination ligand(s) correspond(s) to formula XI:

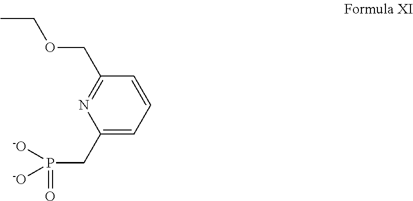

34. The MRI contrast agent according to claim 15, wherein the coordination ligand(s) correspond(s) to formula I: 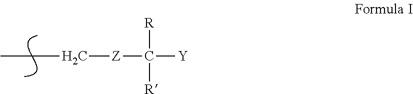
wherein R and R′ are independently hydrogen or an alkyl chain,
Z is NR or O,
Y is CO₂⁻, PO₃²⁻ or SO₃²⁻.

35. Method of acquiring an image, comprising:
a) the administration of the contrast agent according to claim 15 to a tissue, cell or patient, and
b) the acquisition of a magnetic resonance image of said cell, tissue or patient.

36. The method according to claim 35, comprising administering a composition comprising said contrast agent and a pharmaceutically acceptable carrier.
